(19) United States
(12) Patent Application Publication
(10) Pub. No.: US 2006/0013838 A1
(43) Pub. Date: Jan. 19, 2006

(54) Cosmetic powder compositions having large particle size color travel effect pigments

(76) Inventors: Qinyun Peng, Yorktown Heights, NY (US); Philip Linz, Croton-Hudson, NY (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/889,003

(22) Filed: Jul. 13, 2004

(51) Int. Cl. A61K 8/29; A61K 8/25

(52) U.S. Cl. 424/401; 424/63

(57) ABSTRACT

A cosmetic composition preferably in powder form comprising pigments having a color travel effect, wherein said composition contains a sufficient amount of large particle size color travel pigments having a D50 particle size of at least 40 μm up to about 150 μm to retain the color travel effect upon application to skin.

This invention relates to cosmetic compositions preferably powder compositions having color travel effects.

With the emerging of new pigment technologies, interference pigments with unique, spectacular effects, such as color travel (“color variable”), have been developed. However, the eye-catching color travel pigments of normal particle sizes (1-80 μm) is not readily visible in cosmetic powder products or upon application on the skin when a relatively low concentration of the pigments was used. By low concentration is meant generally about 1-10 percent by weight in the powder composition. If a large quantity of the normal particle size color travel pigments is used, it is likely possible to achieve the color travel effect, but it may not be economically practical with this type of special effect pigments.

An objective of this invention, therefore, is to provide an economical cosmetic formulation having a color travel effect.

Upon further study of the specification, other objects and advantages of the invention will become apparent.

To achieve these objects, there are provided cosmetic compositions preferably in powder form, comprising color travel pigments, wherein said composition contains a sufficient amount of large particle size color travel pigments to retain the color travel effect in the formulations and upon application to skin.

By using large particle size color travel pigments, it is possible to achieve the color travel effect on the skin using powder applications and the like without using high concentrations of the pigments, unlike their normal particle size counterparts. Here, “large particle size” pigments are defined as having a median particle size (D50) of 40 μm or higher, preferably 60 μm or higher. The normal particle size pigments usually have median particle sizes (D50) less than 40 μn but larger than 5 μm.

The maximum D50 of the large particle size pigments is preferably 150 μm, more preferably about 85 μm with the preferred particle size D50 being 75-95 μM. The particle size of such glitter pigment can reach up to about 250 μm.

One example of the normal particle size pigment is silica based color travel pigments. When 10% of this type of pigment (its particle size range is 10-50 μm, D50 is 16-25 μm) is added to powder formulations, a subtle color travel effect can be seen from the powder cake, but it disappears once the powder is applied onto the skin. On the other hand, if a color travel pigment with large particle size (D50>60 μm) is employed, not only the color travel effect can be observed clearly on the powder cake, but it is also retained upon application on the skin. Without being bound by an explanation of the unexpected advantage of using large particle size color travel pigments in powder formulations and their application on skin, at least two factors are believed to be important:

The Presence of Light Scattering Agents:

In a typical powder formulation, talc, kaolin, starch, and magnesium stearate are usually used at high concentration (more than 60% by weight combined, in many cases). Absorption pigments are also commonly used to impart colors (e.g. FD&C colors, iron oxides, etc.). The particle sizes of the fillers and absorption pigments are generally less than 25 μm. Their functions are filling, anti-caking or imparting colors. However, they also partially act as light scattering agents due to their high edge to area ratio. In some cases, they may also inevitably decorate the surface of the color travel pigments and consequently reduce the light reflection from the surface of the color travel pigments. The scattering effect (or morphological perturbation) from other ingredients in powder formulations usually reduces or eliminates the color travel effect. However, it has less impact on large particle size pigments and therefore, the luster intensity of large particle size color travel pigment decreases less significantly than that of smaller particle size color travel pigment. The incorporation of at about 1-30% preferably less than 25%, more preferably about 5-20% by weight of the large color travel pigment particles is generally sufficient to obtain the desired color travel effect, but the specific value may vary dependent on the ingredients of the powder composition. The upper limit of the concentration of the large particles will be dictated by aesthetic and cost factors.

Skin Property and Pigment Orientation:

Many skin topology studies have shown that human skin is not completely smooth. It has invisible peaks and valleys, i.e. skin roughness. Some skin roughness data were reported according to different testing methods. They range from a few micrometers to tens of micrometers depending on the testing methods, skin conditions and many other factors. Nonetheless, the skin roughness can be significant enough to interfere with the pigment performance on the skin.

When a powder product is applied onto the skin, the reflectivity of color travel pigment can be further reduced by the skin roughness due to the disorientation of pigment particles and light scattering from the skin. As indicated above, the effect of light scattering from the skin on the large particle size pigments is less pronounced than on the small particle pigments. Hence, our eyes perceive a higher brilliance from the large particle size pigments. Furthermore, large particle size color travel pigments can generally align themselves in parallel better than the smaller ones, owing to their flow characteristics or higher aspect ratio. This again renders a less disturbed light reflection and consequently higher luster intensity and more visible color from the large particle size color travel pigments.

Possible Compositions of the Color Travel Pigments:

One type of color travel pigments described in the present invention are substrate-based pearlescent pigments. Suitable base substrates for the inventive pigments according to the invention are flake-form substrates. Preferred substrates are phyllosilicates. Particularly suitable are natural and/or synthetic mica, aluminum oxides, glass flakes, SiO₂ flakes, talc, kaolin, sericite, flake-form iron oxides or TiO₂ flakes, graphite flakes, BiOCl or other comparable materials.

The size of the base substrates is important per se and can be matched to the particular application. In general, the flake-form substrates have a thickness of between 0.05 and 5 μm, in particular between 0.1 and 4.5 μm. The size in the other two directions is usually between 1 and 550 μm, preferably between 2 and 300 μm, and in particular between 10 and 150 μm (at least 75% within range). The aspect ratio (ratio of surface dimension to thickness of an object) is preferably about 1-500, especially 40-350.

Pigments having color travel effects are defined as exhibiting angle-dependent color change between a number of intense interference colors.

The color travel pigments according to the invention have high and/or low refractive-index layer(s) on top of the surface. The high-refractive-index layer(s) have a refractive index of n>1.8, preferably of n>2.0. The high refractive-index layers preferably comprise TiO₂, ZrO₂, SnO₂, ZNO, BiOCl, Fe₂O₃, Fe₃O₄, Cr₂O₃, CeO₃, molybdenum oxides, CoO, CO₃O₄, VO₂, V₂O₃, NiO, V₂O₅, CuO, Cu₂O, Ag₂O, CeO₂, MnO₂, Mn₂O₃, Mn₂O₅, titanium oxynitrides, pseudobrookite, ilmenite, as well as titanium nitride, MoS₂, WS₂ or mixtures or combinations thereof. The TiO₂ here can be in the rutile or anatase modification, preferably in the rutile modification.

Suitable low-refractive-index materials (n<1.8) are preferably metal oxides or the corresponding oxide hydrates, such as, for example, SiO₂, Al₂O₃, AlO(OH), B₂O₃, MgF₂, MgSiO₃ or a mixture of the said metal oxides.

Particularly interesting color travel pigments have the following layer sequences:
substrate+TiO₂+SiO₂+TiO₂
substrate+TiO₂+SiO₂+TiO₂+absorption pigments or water insoluble dye(s)/lake(s) (such as ferric ferrocyanide, carmine, FD&C dyes/lakes or D&C dyes/lakes, etc.)

The pigments according to the invention can be prepared relatively easily by the precipitation of high- and low-refractive-index metal oxide layers having precisely defined thickness and a smooth surface on the finely divided, flake-form substrates.

The metal-oxide layers are preferably applied by wet-chemical methods. Methods of this type are described, for example, in DE 14 67 468, DE 19 59 988, DE 20 09 566, DE 22 14 545, DE 22 15 191, DE 22 44 298, DE 23 13 331, DE 25 22 572, DE 31 37 808, DE 31 37 809, DE 31 51 343, DE 31 51 354, DE 31 51 355, DE 32 11 602, DE 32 35 017 or in other patent documents and other publications known to the person skilled in the art.

In the wet coating method, the substrate particles are suspended in water, and one or more hydrolyzable metal salts are added at a pH which is suitable for hydrolysis and which is selected so that the metal oxides or metal oxide hydrates are precipitated directly onto the flakes without secondary precipitations occurring. The pH is usually kept constant by simultaneous metered addition of a base or acid. The pigments are subsequently separated off, washed and dried and, if desired, calcined, where the calcination temperature can be optimized with respect to the coating present in each case. In general, the calcination temperatures are between 250 and 1000° C., preferably between 350 and 900° C. If desired, the pigments can be separated off after application of individual coatings, dried and, if desired, calcined and then re-suspended for the deposition of the further layers.

The coating may furthermore also take place in a fluidized-bed reactor by gas-phase coating, it being possible, for example, to use correspondingly the methods proposed in EP 0 045 851 and EP 0 0106 235 for the preparation of color travel pigments.

The production of Ti suboxide or Fe₃O₄ layers can be carried out, for example, by reduction of the TiO₂ layer using ammonia, hydrogen and also hydrocarbons and hydrocarbon/ammonia mixtures, as described, for example, in EP-A-0 332 071, DE 199 51 696 A1 and DE 199 51 697 A1. The reduction is preferably carried out in a forming-gas atmosphere (92% of N₂/8% of H₂ or 96% of N₂/4% of H₂). The reduction is generally carried out at temperatures of 250-1000° C., preferably 350-900° C. and in particular 500-850° C.

The hue of the pigments can be varied within broad limits through a different choice of the coating amounts or the layers resulting therefrom. Fine tuning for a certain hue can be achieved beyond the pure choice of amount by approaching the desired color under visual or measurement technology control.

Furthermore, organic or combined organic/inorganic post-coatings are possible, for example with silanes, as described, for example, in EP 0090259, EP 0 634 459, WO 99/57204, WO 96/32446, WO 99/57204, U.S. Pat. No. 5,759,255, U.S. Pat. No. 5,571,851, WO 01/92425 or in J. J. Ponjeé, Philips Technical Review, Vol. 44, No. 3, 81 ff. and P. H. Harding J. C. Berg, J. Adhesion Sci. Technol. Vol. 11 No. 4, pp. 471-493.

The pigments of the present invention can also advantageously be used in blends with organic dyes, organic pigments or other pigments, such as, for example, transparent and opaque white, colored and black pigments, and with flake-form iron oxides, organic pigments, holographic pigments, LCPs (liquid crystal polymers) and conventional transparent, colored and black luster pigments based on metal oxide-coated mica and SiO₂ flakes, etc. The color travel pigments can be mixed in any ratio with commercially available pigments and fillers.

As for the nature of the color travel pigments, all types which exhibit a color travel effect can be used in the present invention. More examples of such pigments include but are not limited to those described in published U.S. patent application Ser. No. 10/608,563, by Cristoph Schmidt et al. filed Jun. 30, 2003, as well as to those described in the patents and literature cited therein, e.g. U.S. Pat. No. 4,434,010, JP H7-759, U.S. Pat. No. 3,438,796, U.S. Pat. No. 5,135,812, DE 44 05 494, DE 44 37 753, DE 195 16 181 and DE 195 15 988, DE 196 18 565, DE 197 46 067 and in the literature, for example in EURO COSMETICS, 1999, No. 8, p. 284.

The compositions of this invention are primarily in the form of a cosmetic powder for application to skin. Examples of such cosmetic powders include but are not limited to: eye shadow, blusher, powder makeup, lip powder, face powder, body powder, bronzing powder.

Aside from powders, it is contemplated that the powders can be incorporated in various systems so as to form formulations such as for example foundation (liquid and stick), face makeup such as cream-to-powder, eye highlighter, eye pencil, bronzing stick, etc.

Also, the large particle size color travel pigments of this invention will exhibit a sparkling effect and appear to be more lustrous than the normal particle size color travel pigments when dispersed in a wax base or fluid system or the like, such as, for example, in lip gloss, lipstick, nail polish, eyeliner, mascara, hair gel, shower gel, body lotion, skin cream, shampoo, etc.

To reiterate, important aspects of this invention, include but are not limited to color travel pigments having a large particle size (D50>60 μm), and coated with two layers or more, especially with alternating layers of high and low refractive index, for example, with TiO₂—SiO₂—TiO₂. The preferred coatings are with metal oxides, preferably TiO₂ and SiO₂ and/or Fe₂O₃, with the TiO₂ being rutile or anatase, preferably rutile. The large particle size color travel pigments of the invention can be modified with a coating of absorption pigments or water insoluble dye/lakes on top, for example without exclusion ferric ferrocyanide, Indigo, Carmine, FD&C dyes and lakes, and D&C dyes and lakes. A protective layer can also be provided on the modified or unmodified color travel pigment. The substrate of the color travel pigments include, but are not limited to: mica, SiO₂ flakes, Al₂O₃ flakes, glass flakes, graphite flakes, and BiOCl.

The desired particle sizes of the pigments are obtained by conventional methods, e.g. sieving or sedimentation.

The color travel pigments and pigment mixtures have particular applications in decorative and personal care cosmetic preparations, especially in powder form.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

As a disclaimer, it is to be noted that one or more examples may have not been actually conducted.

### EXAMPLES 1-4

LOOSE EYE SHADOW POWDER WITH COLOR
TRAVEL PIGMENTS

| | TRADE | |
|---|---|---|
| INCI NAME | NAME/MANUFACTURER | % by weight |
| Phase A | | |
| Talc | Supra H/Luzenac | q.s. to 100.00 |
| Kaolin | Kaolin, Colloidal, USP/Whittaker, | 4.0 |
| | Clark & Daniels | |
| Magnesium | Magnesium Stearate Vegetable | 2.00 |
| Stearate | F. G./Whittaker, Clark & Daniels | |
| Bismuth | Biron ® LF-2000/Rona | 5.00 |
| Oxychloride | | |
| Phase B | | |
| Iron Oxides | Colorona ® Aborigine Amber/Rona | 7.00 |
| (and) Mica | | |
| | Color Travel Pigment (see below) | |
| Phase C | | |
| Octyldodecanol | Eutanol G/Cognis | 5.00 |
| Total | | 100.00 |

Procedure: Combine Phase A. Add Phase B with gentle agitation. Spray Phase C onto batch while agitating bulk. Pass entire batch through a jump gap.

Several examples are prepared, substituting the specific color travel pigment into the above formula:

| Example | Wt % | *Description | D50 |
|---|---|---|---|
| 1 | 25% | Large particle size color travel (Red/Gold) | ˜86 |
| 2 | 25% | Xirona ® Magic Mauve | 16-22 |
| 3 | 10% | Large particle size color travel (Red/Gold) | ˜86 |
| 4 | 10% | Xirona ® Magic Mauve | 16-22 |

INCI Name:

Large particle size color travel (Red/Gold): mica (and) titanium dioxide (and) silica (and) tin oxide
Xirona® Magic Mauve: silica (and) titanium dioxide (and) tin oxide

### EXAMPLES 5-8

EYE SHADOW WITH COLOR TRAVEL PIGMENTS

| | TRADE | |
|---|---|---|
| INCI NAME | NAME/MANUFACTURER | % |
| Phase A | | |
| Talc | Supra H/Luzenac | q.s. to 100.00 |
| Aluminium | Dry Flo PC/National Starch & | 7.30 |
| Starch Octenyl | Chemical | |
| Succinate | | |
| Magnesium | Magnesium Stearate Vegetable | 2.45 |
| Stearate | F. G./Whittaker, Clark & Daniels | |
| Bismuth | Biron ® LF-2000/Rona | 7.00 |
| Oxychloride | | |
| Phase B | | |
| Iron Oxides | Colorona ® Aborigine Amber/Rona | 7.00 |
| (and) Mica | | |
| | Color Travel Pigment (see below) | |
| Phase C | | |
| Isopropyl | Lexol 3975/Inolex | 10.50 |
| Palmitate (and) | | |
| Isopriopyl | | |
| Myristate (and) | | |
| Isopropyl Sterate | | |
| Cetyl Palmitate | Cutina CP/Henkel | 0.70 |
| Petrolatum | Snow White Petrolatum/Penreco | 0.70 |
| Propylparaben | Propylparaben/Spectrum Chemical | 0.10 |
| Total | | 100.00 |

Procedure: Combine ingredients in Phase A. Pulverize with a hammer mill, passing twice through a 0.27″ herring bone screen. Add Phase B with gentle agitation. Combine Phase C; heat to 70° C. Solution should be clear and uniform. Spray Phase C onto batch while agitating bulk. Pass entire batch through a jump gap.

Several examples are prepared, substituting the specific color travel pigment into the above formula:

| Example | Wt % | *Description | D50 |
|---|---|---|---|
| 5 | 25% | Large particle size color travel (Red/Gold) | ˜86 |
| 6 | 25% | Xirona ® Magic Mauve | 16-22 |
| 7 | 10% | Large particle size color travel (Red/Gold) | ˜86 |
| 8 | 10% | Xirona ® Magic Mauve | 16-22 |

INCI Name:

Large particle size color travel (Red/Gold): mica (and) titanium dioxide (and) silica (and) tin oxide
Xirona® Magic Mauve: silica (and) titanium dioxide (and) tin oxide

The above examples showed that when 10% of normal particle size color travel pigment is used, the color travel effect is merely visible in the powder samples and not visible upon application onto skin. However, the color travel effect can be seen clearly in the powder samples containing the large particle size color travel (Red/Gold) pigment and when the powders are applied onto skin.

In the following non-limiting examples 9-18, the invention can be realized by adding or substituting a sufficient amount of any of the above exemplified large particle size color travel pigments having a D50 particle size of at least 40 μm, preferably at least 60 μm, to each product. For example, the eye highlighter of example 14 can be made by adding 25% by weight of large particle size color travel (Red/Gold) having a D50 of about 86. The remaining components of example 14 will then amount to 75% by weight of the Eye Highlighter.

### EXAMPLE 9

4-IN-1 POWDER MAKEUP
(FOR EYES, CHEEKS, LIPS AND NAILS)
WITH PEARLESCENT PIGMENTS

| INCI NAME | TRADE NAME/MANUFACTURER | % |
|---|---|---|
| Phase A | | |
| Mica (and) Titanium | Pearlescent Pigments/Rona | 35.00 |
| Dioxide; may contain | (see note below) | |
| Iron Oxides (and) Silica | | |
| Bismuth Oxychloride | Biron ® LF-2000/Rona | 5.00 |
| Talc | Supra H/Luzenac | 38.00 |
| Kaolin | Kaolin, Colloidal, USP/Whittaker, | 5.00 |
| | Clark & Daniels | |
| Magnesium Stearate | Magnesium Stearate Vegetable F. G./ | 2.50 |
| | Whittaker, Clark & Daniels | |
| Calcium Silicate | Hubersorb 600/Huber | 0.50 |
| Phase B | | |
| Mineral Oil (and) | Amerchol L-101/Amerchol | 11.90 |
| Lanolin Alcohol | | |
| Lanolin Alcohol | Super Hartolan/Croda | 1.00 |
| Petrolatum | Snow White Petrolatum/Penreco | 1.00 |
| Propylparaben | Propylparaben/Spectrum Chemical | 0.10 |
| Total | | 100.00 |

Procedure: Mix the ingredients of Phase A homogeneously. Combine ingredients in Phase B and heat to 80° C. with mixing. Add the melted Phase B to Phase A with stirring. The powder is pressed at 40-50 bar (˜600-700 lb./sq. in.).
Notes:
A wide range of shades can be made using Xirona ® Color travel pigments, Timiron ® silver, gold, or interference pigments or the colored Colorona ® pigments, either by themselves or in combination. The INCI name may vary, depending on the specific pigment used.

Application: Apply to eyelids and cheeks and blend gently with fingertips. Outline lips with contour pencil and fill in with color. For nails, apply powder to nails with fingertips and fix with clear nail lacquer. Excess powder on cuticles can easily be washed off.

### EXAMPLE 10

FOUNDATION

| | TRADE | |
|---|---|---|
| INCI NAME | NAME/MANUFACTURER | % |
| Phase A | | |
| Water | Water, Demineralized | 50.32 |
| Hydroxylated Lecithin | Alcolec Z-3/American Lecithin | 0.10 |
| | Co. | |
| Phase B | | |
| Mica | Mica M/Rona | 5.00 |
| Titanium Dioxide (and) Mica | Microna ® Matte White/Rona | 4.00 |
| (and) Zinc Oxide | | |
| Mica (and) Iron Oxides | Microna ® Matte Yellow/Rona | 2.00 |
| Mica (and) Iron Oxides | Microna ® Matte Orange/Rona | 0.20 |
| Iron Oxides (and) Mica | Microna ® Matte Red/Rona | 0.20 |
| Iron Oxides (and) Mica | Microna ® Matte Black/Rona | 0.20 |
| Phase C | | |
| Propylene Glycol | Propylene Glycol/Lyondell | 4.00 |
| Magnesium Aluminum | Veegum/RT Vanderbilt | 1.00 |
| Silicate | | |
| Phase D | | |
| Propylene Glycol | Propylene Glycol/Lyondell | 4.00 |
| Cellulose Gum | CMC 7H3SF/Aqualon | 0.15 |
| Phase E | | |
| Deionized Water | Deionized Water | 5.00 |
| Triethanolamine 99% | Triethanolamine 99%/Dow | 1.00 |
| Phase F | | |
| Sucrose Cocoate | Crodesta SL40/Croda | 1.50 |
| Methyl Paraben | Methylparaben/Spectrum | 0.20 |
| | Chemical | |
| Disodium EDTA | Hamp-ene Na2/Hampshire | 0.05 |
| Phase G | | |
| Propylene Glycol Dicaprylate | Myritol PC/Cognis | 11.00 |
| Dicaprate | | |
| Isostearyl Stearoyl Stearate | Hetester ISS/Heterene | 2.00 |
| Sorbitan Monolaurate | Arlacel 20/Uniquema | 2.50 |
| Cetyl Alcohol | Crodacol C-70/Croda | 1.25 |
| Stearic Acid | Emersol 120/Cognis | 1.50 |
| Oleic Acid | Emersol 210/Cognis | 0.50 |
| BHA | Tenox BHA/Eastman | 0.05 |
| Propyl Paraben | Propylparaben/Spectrum | 0.10 |
| | Chemical | |
| Phase H | | |
| DMDM Hydantoin 55% | Glydant/Lonza | 0.18 |
| Water | Water, Demineralized | 2.00 |
| Total | | 100.00 |

Procedure:

Water Phase: Combine the ingredients of Phase A. Mix until homogenous. Add Phase B with stirring, mixing until no undispersed pigment remains. Combine and add Phase C. Continue mixing and heat to 90° C. for 15 minutes. Cool to 75° C. Combine and add Phase D and Phase E, maintaining agitation until no undispersed particles remain. Add Phase F.

Oil Phase: Combine the ingredients of phase G separately from the water phase. Heat to 75-80° C. with propeller agitation until homogenous.

Emulsification: Add the oil phase to the water phase at 75-80° C., with propeller agitation. Maintain temperature and homogenize for 15 minutes. Cool to 45° C. with moderate agitation. Combine and add Phase H. Cool to 30° C.

### EXAMPLE 11

SPF 4.5 LOOSE BODY POWDER

| INCI Name | Trade Name (Supplier) | % w/w |
|---|---|---|
| Phase A | | |
| Talc | Supra H/Luzenac America | 40.50 |
| Mica | Mica M/Rona | 27.50 |
| Mica (and) Silica | Micronasphere ® M/Rona | 10.00 |
| Kaolin | Kaolin, Colloidal, USP/ | 4.00 |
| | Whittaker, Clark & Daniels | |
| Magnesium Stearate | Magnesium Stearate Vegetable | 5.00 |
| | F. G./Whittaker, Clark & Daniels | |
| Titanium Dioxide (and) | Eusolex ® T-2000/Rona | 5.00 |
| Alumina (and) Simethicone | | |
| Mica (and) Iron Oxides | Microna ® Matte Orange/Rona | 4.34 |
| Iron Oxides (and) Mica | Microna ® Matte Red/Rona | 0.84 |
| Mica (and) Iron Oxides | Microna ® Matte Yellow/Rona | 0.84 |
| Mica (and) Iron Oxides | Microna ® Matte Black/Rona | 0.98 |
| Titanium Dioxide (and) | Microna ® Matte White/Rona | 1.00 |
| Mica (and) Zinc Oxide | | |
| Total | | 100.00 |

Procedure: The powder ingredients are mixed homogeneously and then sieved through a 63 μm screen.
Note:
In vivo SPF 4.5 (5 subjects), Colipa Task Force Method

### EXAMPLE 12

LIP POWDER

| INCI NAME | TRADE NAME/MANUFACTURER | % |
|---|---|---|
| Phase A | | |
| Mica (and) Iron | Colorona ® Bordeaux/Rona | 30.00 |
| Oxides | | |
| Bismuth | Biron ® LF-2000 or Biron ® MTU/Rona | 10.00 |
| Oxychloride | | |
| Talc | Supra Talc/Luzenac | 40.00 |
| Magnesium | Magnesium Stearate Vegetable Food Grade/ | 5.00 |
| Stearate | Whittaker, Clark & Daniels | |
| Phase B | | |
| Isopropyl Stearate | Tegosoft S/Goldschmidt | 11.20 |
| Dimethicone | 1403 Fluid/Dow Coming | 3.75 |
| (and) | | |
| Dimethiconol | | |
| Fragrance | Fragrance | q.s. |
| Propylparaben | Propylparaben/Sutton | 0.05 |
| Total | | 100.00 |

Procedure: Mix the ingredients of Phase A homogeneously. Heat and mix the ingredients in Phase B. Add Phase B to Phase A with mixing. The powder is pressed between 40-50 bar.

### EXAMPLE 13

EYE PENCIL

| INCI NAME | TRADE NAME/MANUFACTURER | % |
|---|---|---|
| Phase A | | |
| Italian Talc | Supra H/Luzenac | 11.30 |
| C18-36 Ethylene Glycol | Syncrowax ERL-C/Croda | 8.10 |
| Ester | | |
| Glyceryl Tribehenate | Syncrowax HR-C/Croda | 1.90 |
| Ethyl Hexyl Palmitate | Pelemol OP/Phoenix Chemical | 38.40 |
| Caprylic/Capric | Myritol 318/Cognis | 4.00 |
| Triglyceride | | |
| Stearic Acid | Emersol 120/Cognis | 3.00 |
| Polyglyceryl 3- | Emerest 2452/Cognis | 0.50 |
| Diisostearate | | |
| Methylparaben | Methylparaben/Spectrum Chemical | 0.20 |
| Propylparaben | Propylparaben/Spectrum Chemical | 0.10 |
| Phase B | | |
| Bismuth Oxychloride | Biron ® MTU/Rona | 15.00 |
| Iron Oxides (and) Mica | Microna ® Matte Red/Rona | 16.00 |
| Iron Oxides (and) Mica | Microna ® Matte Black/Rona | 1.50 |
| Total | | 100.00 |

Procedure: Combine all ingredients in Phase A and heat to 80-85° C. with stirring until homogeneous. Add Phase B. Agitate with a high-speed mixer until no agglomerates remain. Pour at 70° C.

### EXAMPLE 14

EYE HIGHLIGHTER

| INCI NAME | TRADE NAME/MANUFACTURER | % |
|---|---|---|
| Phase A | | |
| Talc | Supra H/Luzenac | 48.25 |
| Starch | Novation 1600/National Starch | 7.30 |
| | & Chemical | |
| Magnesium Stearate | Magnesium Stearate Vegetable | 2.45 |
| | F. G./WC&D | |
| Bismuth Oxychloride | Biron ® LF-2000/Rona | 5.70 |
| Phase B | | |
| Mica (and) Titanium | Timiron ® MP-99/Rona | 17.00 |
| Dioxide | | |
| Mica (and) Titanium | Colorona ® Magenta/Rona | 4.90 |
| Dioxide (and) Carmine | | |
| Mica (and) Titanium | Colorona ® Red Gold/Rona | 2.40 |
| Dioxide (and) Iron | | |
| Oxides | | |
| Phase C | | |
| Isopropyl Stearate | Tegosoft S/Goldschmidt | 10.50 |
| Cetyl Palmitate | Cutina CP/Cosgnis | 0.70 |
| Petrolatum | Snow White Petrolatum/Penreco | 0.70 |
| Propylparaben | Propylparaben/Spectrum Chemical | 0.10 |
| Total | | 100.00 |

Procedure: Combine Phase A. Pulverize with a hammer mill, passing twice through a 0.027″ herring bone screen. Add Phase B with gentle agitation. Combine Phase C. Heat to 70° C. Spray onto batch while agitating bulk. Pass entire batch through a jump gap.

### EXAMPLE 15

BRONZING POWDER

| INCI NAME | TRADE NAME/MANUFACTURER | % |
|---|---|---|
| Phase A | | |
| Talc | Supra H/Luzenac | q.s. to |
| | | 100.00 |
| Lithium Stearate | Lithium Stearate #306/Witco | 3.50 |
| Kaolin | Kaolin 2457/Whittaker, Clark & | 5.00 |
| | Daniels | |
| Calcium Silicate | Microcel E/Celite | 0.30 |
| Methylparaben | Methylparaben/Spectrum Chemical | 0.20 |
| Propylparaben | Propylparaben/Spectrum Chemical | 0.10 |
| Bismuth Oxychloride | Biron ® MTU/Rona | 5.00 |
| Phase B | | |
| Mica (and) Iron Oxides | Colorona ® Bronze/Rona | 50.00 |
| Phase C | | |
| Isopropyl Palmitate | Lexol IPP/Inolex | 7.30 |
| Cetyl Palmitate | Cutina CP/Cognis | 2.00 |
| Petrolatum | White Petrolatum/Witco | 2.00 |
| Polyglyceryl-3 | Emerest 2452/Cognis | 0.50 |
| Diisostearate | | |
| Phase D | | |
| Fragrance | Grapefruit Fragrance 26125V/Shaw | 0.10 |
| | Mudge | |
| Total | | 100.00 |

Procedure: Combine Phase A. Pulverize with a hammer mill, passing twice through a 0.027″ herring bone screen. Add Phase B with gentle agitation. Combine Phase C. Heat to 70° C., stirring until clear. Reduce temperature to 55-60° C. Add Phase D (if desired). Spray onto batch while agitating bulk. Pass entire batch through a jump gap.

### EXAMPLE 16

FACE POWDER

| INCI NAME | TRADE NAME/MANUFACTURER | % |
|---|---|---|
| Talc | Supra H/Luzenac America | 87.10 |
| Aluminum Starch | Dry Flo Pure/National Starch | 11.00 |
| Octenylsuccinate | | |
| Mica (and) Iron Oxides | Microna ® Matte Yellow/Rona | 1.00 |
| Mica (and) Iron Oxides | Microna ® Matte Orange/Rona | 0.20 |
| Iron Oxides (and) Mica | Microna ® Matte Red/Rona | 0.20 |
| Iron Oxides (and) Mica | Microna ® Matte Black/Rona | 0.20 |
| Methylparaben | Methylparaben/Spectrum Chemical | 0.10 |
| Propylparaben | Propylparaben/Spectrum Chemical | 0.10 |
| Imidazolidinyl Urea | Germall 115/Sutton | 0.10 |
| Total | | 100.00 |

Procedure: Add ingredients to powder blender. Mix until uniform. Micropulverize. Package.

### EXAMPLE 17

CREAM TO POWDER MAKEUP

| INCI NAME | TRADE NAME/MANUFACTURER | % |
|---|---|---|
| Phase A | | |
| Silica (and) Titanium Dioxide | Ronasphere ® LDP/Rona | 10.00 |
| (and) Iron Oxides | | |
| Mica (and) Titanium Dioxide | Extender W/Rona | 12.00 |
| Mica (and) Iron Oxides | Microna ® Matte Yellow/Rona | 4.40 |
| Iron Oxides (and) Mica | Microna ® Matte Red/Rona | 0.80 |
| Iron Oxides (and) Mica | Microna ® Matte Black/Rona | 0.40 |
| Mica (and) Iron Oxides | Microna ® Matte Orange/Rona | 0.40 |
| Talc | Supra H/Luzenac America | 6.40 |
| Silica | Ronasphere ®/Rona | 2.10 |
| Aluminum Starch Octenyl Succinate | Dry Flo PC/National Starch & Chemical | 14.40 |
| Phase B | | |
| Octyl Palmitate | Pelemol OP/Phoenix | 29.50 |
| Mineral Oil (and) Lanolin Alcohol | Amerchol L-101/Amerchol | 2.50 |
| Copernica Cerifa (Carnauba) Wax | Carnauba Wax/Ross | 1.30 |
| Ceresin | Ceresin Wax #375/Ross | 2.00 |
| Hydrogenated Castor Oil | Cutina HR/Henkel | 3.50 |
| Phenyl Trimethicone | Dow Corning 556/Dow Corning | 9.50 |
| Sorbitan Sesquioleate | Arlacel 83/Uniquema | 0.50 |
| Propylparaben | Propylparaben/Spectrum Chemical | 0.20 |
| PEG-8 (and) Tocopherol (and) Ascorbyl | Oxynex ® K/Rona | 0.10 |
| Palmitate (and) Ascorbic Acid (and) | | |
| Citric Acid | | |
| Total | | 100.00 |

Procedure:

Heat the ingredients of Phase B to 85° C. with mixing. Blend Phase A and add to Phase B. Continue mixing until the melt is homogeneous. Cool down to 80° C. and pour into molds.

### EXAMPLE 18

BLUSHER

| | TRADE NAME/ | |
|---|---|---|
| INCI NAME | MANUFACTURER | % |
| Phase A | | |
| Octyl Palmitate | Pelemol OP/Phoenix | 29.00 |
| | Chemical | |
| Ozokerite | Ozokerite Wax 77W/Ross | 4.00 |
| Hydrogenated Castor Oil | Cutina HR/Cognis | 4.00 |
| Copernica Cerifa | Carnauba Wax/Ross | 1.00 |
| (Carnauba) Wax | | |
| Phenyl Trimethicone | Dow Corning 556 Fluid/Dow | 8.50 |
| | Corning | |
| Mineral Oil (and) | Amerchol L-101/Amerchol | 2.50 |
| Lanolin Alcohol | | |
| Sorbitan Sesquioleate | Arlacel 83/Uniquema | 0.50 |
| Propylparaben | Propylparaben/Spectrum | 0.20 |
| | Chemical | |
| Allantoin | Allantoin/Rona | 0.20 |
| PEG-8 (and) Tocopherol (and) | Oxynex ® K/Rona | 0.10 |
| Ascorbyl Palmitate (and) | | |
| Ascorbic Acid (and) | | |
| Citric Acid | | |
| Phase B | | |
| Talc | Supra H/Luzenac America | 18.60 |
| Aluminum Starch Octenyl | Dry Flo PC/National Starch & | 14.90 |
| Succinate | Chemical | |
| Bismuth Oxychloride | Biron ® MTU/Rona | 12.50 |
| Phase C | | |
| Mica (and) Iron Oxides | Colorona ® Bordeaux/Rona | 3.00 |
| Mica (and) Titanium Dioxide | Colorona ® Carmine Red/Rona | 1.00 |
| (and) Carmine | | |
| Total | | 100.00 |

Procedure: Combine all ingredients in Phase A. Heat to 80-85° C. and mix until homogenous. Add Phase B and Phase C with mixing. Pour at 70° C.

These cosmetic compositions can be applied to skin in the conventional manner.

The preceding examples can be repeated with similar success by substituting the generically or specifically described ingredients and/or operating conditions of this invention for those used in the preceding examples.

The entire disclosures of all applications, patents and publications, cited herein are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

1. A cosmetic powder composition comprising pigments having a color travel effect, wherein said composition contains a sufficient amount, about 1-30% by weight, of large particle size color travel pigments having a D50 particle size of at least 40 μm up to about 150 μm to retain the color travel effect upon application to skin.

2. A cosmetic powder composition according to claim 1, wherein the color travel pigments have a D50 particle size of at least 60 μm.

3. A composition according to claim 1, containing less than 25% by weight of the large particle size color travel pigments.

4. A composition according to claim 1, containing 5-20% by weight of the large particle size color travel pigments.

5. A composition according to claim 2, containing 5-20% by weight of the large particle size color travel pigments.

6. A cosmetic powder composition according to claim 1, wherein the large particle size color travel pigments are coated with alternating layers of high and low refractive index coatings.

7. A coating composition according to claim 6, wherein the layers are metal oxides.

8. A cosmetic composition according to claim 7, wherein the metal oxides are TiO₂ and SiO₂ or Fe₂O₃.

9. A coating composition according to claim 1, wherein the large particle size color travel pigments comprise more than 2 layers of metal oxides.

10. A cosmetic composition according to claim 9, wherein said metal oxide layers are TiO₂—SiO₂—TiO₂.

11. A cosmetic powder composition according to claim 10, wherein the TiO₂ is rutile.

12. A cosmetic powder composition according to claim 1, wherein said color travel pigments comprise a substrate selected from the group consisting of natural/synthetic mica, SiO₂ flakes, Al₂O₃ flakes, glass flakes, graphite flakes and BiOCl, flake-form of TiO₂, flake-form of Fe₂O₃.

13. A cosmetic powder composition according to claim 12, said color travel pigments comprising at least 2 layers of different metal oxides.

14. A cosmetic powder composition according to claim 13, further comprising as a top layer an absorption pigment or a water insoluble dye(s)/lakes(s).

15. A cosmetic powder composition according to claim 14, comprising as a top layer a ferric ferrocyanide, carmine red, indigo, or carmine, FD&C dyes/lakes, D&C dyes/lakes.

16. A color travel pigment having a D50 particle size of at least 40 μm up to about 150 μm.

17. A color travel pigment according to claim 16, having a D50 particle size of at least 60 μm.

18. A color travel pigment according to claim 1, having a D50 range of 75-95 μm.

19. A color travel pigment according to claim 17, wherein said pigment comprises as a top layer an absorption pigment, a water insoluble dye(s) or a water insoluble lake(s).

20. A cosmetic composition comprising pigments having a color travel effect, wherein said composition contains a sufficient amount of large particle size color travel pigments having a D50 particle size of at least 40 μM up to about 150 μm to retain the color travel effect upon application to skin.

21. A cosmetic composition according to claim 20, in the form of an eye pencil or eye highlighter.

22. A cosmetic composition according to claim 20, in the form of a cream to powder.

23. A cosmetic composition according to claim 20, in the form of a liquid or stick foundation or bronzer.

24. A cosmetic composition according to claim 20, in the form of lip gloss, lipstick, nail polish, eyeliner, mascara, hair gel, shower gel, body lotion, skin cream or shampoo.
